(19) United States
(12) Patent Application Publication
(10) Pub. No.: US 2026/0347391 A1
(43) Pub. Date: Nov. 5, 2026

(54) METHOD FOR THE PURIFICATION OF IMMUNOGLOBULINS

(71) Applicant: MABXIENCE RESEARCH, S.L., Madrid (ES)

(72) Inventors: Ivan SANCHEZ MELO, Armunia (ES); Iván LÓPEZ RODRÍGUEZ, Armunia (ES); Jonathan ÁLVAREZ MARTÍNEZ, Armunia (ES)

(21) Appl. No.: 19/487,249

(22) PCT Filed: May 29, 2024
(86) PCT No.: PCT/EP2024/064712

§ 371 Date: Nov. 24, 2025

(30) Foreign Application Priority Data
May 30, 2023 (EP) 23382508.2

(51) Int. Cl. C07K 1/22; C07K 1/18; C07K 1/34; C07K 1/36; C07K 16/28

(52) U.S. Cl. CPC C07K 1/22; C07K 1/18; C07K 1/34; C07K 1/36; C07K 16/2818

(57) ABSTRACT

The present invention relates to a method for the purification of immunoglobulin molecules present in an immunoglobulin preparation, uses and products thereof.

### FIELD OF THE INVENTION

The present invention is related with the field of organic chemistry and methods of preparation of proteins by chromatography, specifically of monoclonal antibodies.

### BACKGROUND OF INVENTION

Protein therapeutics has drastically changed the treatment landscape of many diseases by targeting diseases which either had no real treatment available for opening up new less toxic and more effective avenues of treatment. In particular, there is at the moment a significant increase of monoclonal antibodies (mAbs) entering different stages of development, from screening for new targets to entering the latter stages of clinical trials.

This accelerated development of protein therapeutics requires a production pipeline which is effective keeping the time from production to market at a minimum, while maintaining cost effectiveness and above all safeguarding the quality standards required for human treatment substances. In addition, the different steps in this pipeline must be flexible in order to be adaptable to the different protein characteristics without the need to reorganize the whole of the pipeline.

Protein purification strategies are characterized by following four main steps: 1^{st}: preparation, extraction and clarification; 2^{nd}: capture; 3^{rd}: polishing (which normally includes two operations); and 4^{th}: formulation and final preparation. The first step is the starting point of any purification, the sample preparation, which includes the production of proteins in cell cultures, bacterial lines or viruses engineered to produce said protein of interest. From this culture an extract is obtained and clarified, i.e., cleared of large aggregates and deposits. In the second step, capture, the objective is the isolation, concentration and stabilization of the target protein, in order to obtain the protein in an environment which potentiates its stability and activity, ideally while removing major critical contaminants present in the extract. Next comes polishing, which is performed to obtain the final product at a high degree of purity. In the polishing phase, impurities with similar characteristics and properties as the target protein are removed. Finally, the last step is in charge of leaving the protein in the final formulation and with the final commercial concentration.

Most of the loss of the target protein occurs during the stage of polishing, since in order to obtain the desired purity, contaminants with very similar properties of the target protein have to be eliminated. Despite this, polishing is normally constituted by 2 or more steps of purification, wherein the degree of purification of the obtained product increases with each step as does the protein loss of the target protein. The main method of protein purification during polishing is chromatographic purification, of which several techniques exist, taking advantage of one or more of protein properties such as size, charge, available ligand, isoelectric point, etc.

Multimodal chromatography takes advantage of not one but at least two characteristics of the target protein, interacting with said protein through two or more modes of action, such as electrostatic, hydrophobic and hydrogen bonding interactions, which can be used independently or together. Therefore, multimodal chromatography is a highly flexible technique ideal for the purification of challenging targets. Despite this, certain targets are still unable to be purified at a level required with multimodal chromatography and due to the complexity inherent in the media used for multimodal chromatography, the technique is very time-consuming during process optimization. Therefore, alternative techniques which are simpler to use are required in order to obtain target proteins purified to a higher degree.

### SUMMARY OF THE INVENTION

The present invention discloses a technique of for the purification of a target protein by multimodal chromatography with a dual characteristic resin (dual-resin), ionic and hydrophobic, wherein the elution of the target protein is performed with a pH gradient at constant conductivity. This allows the elution of the target protein with a reduced content of impurities as well as the modulation of the content in charge variants and aggregates. Furthermore, the inventors have found that, surprisingly, by charging the target protein with a charge of the same sign as the resin and thereby decreasing the electrostatic interaction with the dual-resin, the content in charge variants of the target protein could be modulated.

Therefore, a first aspect of the present invention relates to a method for the purification of immunoglobulin molecules present in an immunoglobulin preparation, from here onwards the method of the invention, comprising the following steps:
(i) Adjusting the pH of the preparation to a binding pH value which is different from the pl of the immunoglobulin molecules, thereby providing a net charge to the immunoglobulin molecules;
(ii) Contacting the preparation of (a) with a multimodal ion exchanger at the pH value used in step (i), wherein said multimodal ion exchanger comprises a ligand having an hydrophobic moiety and a moiety which is charged at the binding pH value, thereby allowing the immunoglobulins to bind to the multimodal ion exchanger and
(iii) Eluting the immunoglobulins bound to the multimodal ion exchanger using a decreasing pH gradient at constant conductivity, wherein the moiety is positively charged if the net charge of the immunoglobulin molecules is positive or wherein the moiety is negatively charged if the net charge of the immunoglobulin molecules is negative, thereby obtaining a final purified immunoglobulin preparation.

Another aspect of the present invention relates to an immunoglobulin obtained by the method according to the invention.

A further aspect of the present invention relates to a pharmaceutical composition comprising the immunoglobulin according to the invention or an immunoglobulin obtained by the method according to the invention.

Another aspect of the present invention relates to the immunoglobulin according to the invention or an immunoglobulin obtained by the method of the invention for use in medicine.

Yet another aspect of the present invention relates to the immunoglobulin according to the invention or an immunoglobulin obtained by the method of the invention for use in the treatment of cancer.

One more aspect of the present invention relates to a use of the method according to the invention to reduce the level of high molecular weight aggregates in the immunoglobulin preparation.

Yet another aspect of the present invention relates to a use of the method according to the invention to reduce the contents of charge variants in the immunoglobulin preparation.

A final aspect of the present invention relates to a use of the method according to the invention to reduce the contents of impurities in the immunoglobulin preparation.

### DESCRIPTION OF THE FIGURES

FIG. 1. Downstream overall process yield of the NORMAL versus MULTIMODAL (MM) processes using the MB12 antibody in each step (A) and globally (B). The MM process shows improved yield at MM chromatography step versus the Cation Exchange chromatography (CEX) step of the NORMAL process, as well as a better overall yield performance.

FIG. 2. High Molecular Weight (HMW) aggregates comparison between the NORMAL and ADHERE processes for each step of the processes using the MB12 antibody. Reference Max represents to the maximal desirable HMW aggregates in the final solution. Both processes perform similarly in relation to the removal of HMW aggregates.

FIG. 3. Charge variants Acidic forms by lon exchange chromatography (IEX) analysis for the NORMAL process and the MM process using the MB12 antibody following each step of the method. As can be seen the level of charge variants acidic forms falls between the established reference measurement procedure (RMP) for both.

FIG. 4. Charge variants Basic forms by IEX analysis for the NORMAL process and the ADHERE process using the MB12 antibody following each step of the method. As can be seen the level of charge variants-basic forms falls between the established RMP for both.

FIG. 5. Level of host cell proteins (HCPs) after each step of the purification process using the MB12 antibody. Both the NORMAL and MM processes perform equally well in the elimination of the HCP contaminates.

FIG. 6. Level of host cell DNA after each step of the purification process using the MB12 antibody. Both the NORMAL and MM processes perform equally well in the elimination of DNA contaminates.

FIG. 7. Downstream overall process yield of the NORMAL versus ADHERE (Multimodal, MM) processes using the MB11 antibody in each step (A) and globally (B). The MM process shows improved yield at MM chromatography step versus the Cation Exchange chromatography (CEX) step of the NORMAL process, as well as a better overall yield performance.

FIG. 8. Charge variants Acidic forms by Capillary Zone Electrophoresis (CZE) analysis for the NORMAL process and the ADHERE (Multimodal, MM) process using the MB11 antibody following each step of the method. As can be seen the level of charge variants-acidic forms falls under the established reference measurement procedure (RMP) for both processes.

FIG. 9. Charge variants Basic forms by Capillary Zone Electrophoresis (CZE) analysis for the NORMAL process and the ADHERE (Multimodal, MM) process using the MB11 antibody following each step of the method. As can be seen the level of charge variants basic forms following the ADHERE process falls between the established RMP.

FIG. 10. Level of host cell proteins (HCPs) after each step of the purification process using the MB11 antibody. Both the NORMAL and MM processes perform equally well in the elimination of the HCP contaminates. HCP level is comparable after each step for both process. HCP impurities level after TFF unit operation is similar and comparable to RMP range established.

FIG. 11. Level of host cell DNA after each step of the purification process using the MB11 antibody. Both the NORMAL and MM processes perform equally well in the elimination of the DNA contaminates. DNA level is comparable after each step for both process. HcDNA impurities level after TFF unit operation is similar and comparable to RMP range established.

### DETAILED DESCRIPTION OF THE INVENTION

As previously stated, the present invention discloses a technique of multimodal chromatography using a dual resin, wherein the elution of the target protein is performed with a pH gradient at constant conductivity, which allows, during the elution of the protein, not only to remove impurities but in addition to modulate the charge variants and aggregates. In addition, the present technique uses the same buffer for all the steps of the purification, greatly simplifying the process. Furthermore, the inventors found that, surprisingly, by charging the target protein and decreasing the electrostatic interaction with the ionic resin, they obtained a better modulation of the elution, allowing the modulation of the load variants.

Therefore, a first aspect of the present invention relates to a method for the purification of immunoglobulin molecules present in an immunoglobulin preparation, from here onwards the method of the invention, comprising the following steps:
(i) Adjusting the pH of the preparation to a binding pH value which is different from the pl of the immunoglobulin molecules, thereby providing a net charge to the immunoglobulin molecules;
(ii) Contacting the preparation of (a) with a multimodal ion exchanger at the pH value used in step (i), wherein said multimodal anion exchanger comprises a ligand having an hydrophobic moiety and a moiety which is charged at the binding pH value, wherein the moiety is positively charged if the net charge of the immunoglobulin molecules is positive or wherein the moiety is negatively charged if the net charge of the immunoglobulin molecules is negative, thereby allowing the immunoglobulins to bind to the multimodal ion exchanger and
(iii) Eluting the immunoglobulins bound to the multimodal ion exchanger using a pH gradient at constant conductivity, thereby obtaining a final purified immunoglobulin preparation.

The term “immunoglobulin” or “immunoglobulin molecule”, also known as an “antibody”, as used herein refers to any antigen-binding immunoglobulin fragment or immunoglobulin fusion protein, monoclonal or polyclonal, derived from human or other animal cell lines, including natural or genetically modified forms such as humanized, human, chimeric, synthetic, recombinant, hybrid, mutated, grafted, and in vitro generated antibodies. Commonly known natural immunoglobulin antibodies include IgA (dimeric), IgG, IgE, IgG and IgM (pentameric).

The term “immunoglobulin preparation” in the present context refers to a solution, mix or composition which contains an assortment of immunoglobulin molecules, which can be of different types and/or from different origins, at least one of them is the target of purification of the present method. The preparation comprises as well, other types of molecules and proteins which are generally term contaminants or impurities which ideally are removed from the preparation by the method of the invention. In a particular embodiment of the method of the invention, the immunoglobulin molecules in the preparation consist essentially of immunoglobulin G, preferably immunoglobulin G4. The term “immunoglobulin G” or “IgG” refers to a polypeptide belonging to the class of antibodies that are substantially encoded by a recognized immunoglobulin gamma gene. In humans this class comprises IgG1, IgG2, IgG3, and IgG4, named in order of their abundance in serum (IgG4 being the least abundant). Representing approximately 75% of serum antibodies in humans, IgG is the most common type of antibody found in blood circulation. IgG immunoglobulin molecule consists of four polypeptide chains, composed of two identical 50 kDa y heavy (H) chains and two identical 25 kDa K or A light (L) chains, linked together by inter-chain disulfide bonds. Each heavy chain consists of an N-terminal variable domain (VH) and three constant domains (CH1, CH2, CH3), with an additional “hinge region” between CH1 and CH2. Similarly, the light chains consist of an N-terminal variable domain (VL) and a constant domain (CL). The light chain associates with the VH and CH1 domains to form a Fab arm (“Fab”=fragment antigen binding), and functionally, the V regions interact to form the in antigen-binding. Two heavy chain-light chain heterodimers (HL) combine into a single antibody molecule (H2L2) via disulfide bonds in the hinge region and non-covalent interactions between the CH3 domains. The part of the antibody formed by the lower hinge region and the CH2/CH3 domains is called “Fc” (“fragment crystalline”).

In a particular embodiment of the method of the invention the immunoglobulin G is selected from group consisting of Tremelimumab, Mosunetuzumab, Teclistamab, Donanemab, Spesolimab, Lecanemab, Relatlimab, Faricimab, Tislelizumab, Penpulimab, Sintilimab, Teplizumab, Toripalimab, Omburtamab, Retifanlimab, Sutimlimab, Ublituximab, Inolimomab, Oportuzumab monatox, Narsoplimab, Sotrovimab, Regdanvimab, Casirivimab, imdevimab, Tezepelumab, Tisotumab vedotin, Amivantamab, Anifrolumab, Loncastuximab tesirine, Bimekizumab, Tralokinumab, Evinacumab, Aducanumab, Dostarlimab, Ansuvimab, Margetuximab, Naxitamab, Atoltivimab, Maftivimab, and Odesivimab-ebgn, Belantamab mafodotin, Tafasitamab, Satralizumab, Inebilizumab, Sacituzumab govitecan, Teprotumumab, Isatuximab, Eptinezumab, [fam]-trastuzumab deruxtecan, Enfortumab vedotin, Crizanlizumab, Brolucizumab, Polatuzumab, Risankizumab, Romosozumab, Caplacizumab, Ravulizumab, Emapalumab, Cemiplimab, Fremanezumab, Moxetumomab pasudotox, Galcanezumab, Lanadelumab, Mogamulizumab, Erenumab, Tildrakizumab, Ibalizumab, Burosumab, Durvalumab, Emicizumab, Benralizumab, Ocrelizumab, Guselkumab, Inotuzumab, ozogamicin, Sarilumab, Dupilumab, Avelumab, Brodalumab, Atezolizumab, Bezlotoxumab, Olaratumab, Reslizumab, Obiltoxaximab, Ixekizumab, Daratumumab, Elotuzumab, Necitumumab, Alirocumab, Mepolizumab, Evolocumab, Dinutuximab, Secukinumab, Nivolumab, Blinatumomab, Pembrolizumab, Ramucirumab, Vedolizumab, Siltuximab, Obinutuzumab, Ado-trastuzumab emtansine, Raxibacumab, Pertuzumab, Brentuximab vedotin, Belimumab, Ipilimumab, Denosumab, Tocilizumab, Ofatumumab, Canakinumab, Golimumab, Ustekinumab, Certolizumab pegol, Catumaxomab, Eculizumab, Ranibizumab, Panitumumab, Natalizumab, Bevacizumab, Cetuximab, Efalizumab, Omalizumab, Tositumomab-1131, Ibritumomab tiuxetan, Adalimumab, Alemtuzumab, Gemtuzumab ozogamicin, Trastuzumab, Infliximab, Palivizumab, Basiliximab, Daclizumab, Rituximab, Abciximab, Edrecolomab, Nebacumab, Muromonab-CD3 or any combination thereof.

In another particular embodiment of the method of the invention the immunoglobulin G4 is selected from group consisting of Teclistamab, Relatlimab, Tislelizumab, Sintilimab, Toripalimab, Retifanlimab, Sutimlimab, Narsoplimab, Tralokinumab, Evinacumab, Dostarlimab, Cemiplimab, Galcanezumab, Ibalizumab, Emicizumab, Inotuzumab, Dupilumab, Reslizumab, Ixekizumab, Nivolumab, Pembrolizumab, Natalizumab, Gemtuzumab or any combination thereof. In another particular embodiment of the method of the invention the immunoglobulin G4 is selected from Pembrolizumab or Nivolumab. In the present invention, the antibody Pembrolizumab is also referred to as MB12, while the antibody Nivolumab is also referred to as MB11.

Pembrolizumab, as used herein, refers to a humanized IgG4 which specifically binds and blocks PD1. The sequences of the heavy and light chains are as defined in the ChEMBL database under accession number CHEMBL3137343.

Nivolumab, as used herein, refers to a fully human IgG4 which specifically binds and blocks PD1. The sequences of the heavy and light chains are as defined in the ChEMBL database under accession number CHEMBL2108738.

It will be understood that the terms Pembrolizumab and Nivolumab apply both to the antibodies having heavy chain sequences as defined in the respective ChEMBL database entries as well as to the variants thereof in which the heavy chain C-terminal lysine residue has been cleaved off.

In one embodiment, the method of the invention does not comprise a step of cation exchange chromatography and/or a step of anion exchange chromatography.

### Method of the Invention—First Step

The first step of the method of the invention relates to a process of changing, modifying or adjusting the pH of the immunoglobulin preparation as to obtain a pH value which is ideal for the binding of the target immunoglobulin to the multimodal ion exchanger of the next step. This ideal pH value is the “binding pH value” and it is characterized by being different from the isoelectric point (pI) of the immunoglobulin molecules of interest. The term “isoelectric point” or “pI” as used herein refers to the pH at which a molecule carries no net electrical charge and hence is considered neutral. The net charge on the molecule is affected by pH of its surrounding environment and can become more positively or negatively charged due to the gain or loss, respectively, of protons (H+). Therefore, by lowering or raising the pH of the preparation to below or above, respectively, the pl of the immunoglobulin molecules the immunoglobulins molecules are charged with a positive or negative, respectively, net electrical charge. In a particular embodiment the binding pH of step (i) is below or above the isoelectric point of the immunoglobulin molecules.

A number of algorithms for estimating isoelectric points of peptides and proteins have been developed. Most of them use Henderson-Hasselbalch equation with different pK values (Po and Senozan, 2001 Chem. Educ. 78 (11): 1499-1503). More advanced methods take into account the effect of adjacent amino acids+3 residues away from a charged aspartic or glutamic acid, the effects on free C terminus, as well as they apply a correction term to the corresponding pK values using genetic algorithm (Cargile, et al., 2008, Electrophoresis 29 (13): 2768-2778). Other recent approaches are based on a support vector machine algorithm (Perez-Riverol et al., 2012, Journal of Proteomics 75 (7): 2269-2274) and pKa optimization against experimentally known protein/peptide isoelectric points (Kozlowski, L P. 2016, Biol Direct. 11 (1): 55).

In a particular embodiment of the method of the invention the binding pH differs from the isoelectric point of the immunoglobulins in at least at least 0.1 pH units, at least 0.1 pH units, at least 0.2 pH units, at least 0.3 pH units, at least 0.4 pH units, at least 0.5 pH units, at least 0.6 pH units, at least 0.7 pH units, at least 0.8 pH units, at least 0.9 pH units, at least 1 pH units, at least 1.1 pH units, at least 1.2 pH units, at least 1.3 pH units, at least 1.4 pH units, at least 1.5 pH units, at least 1.6 pH units, at least 1.7 pH units, at least 1.8 pH units, at least 1.9 pH units, at least 2 pH units.

In another particular embodiment of the method of the invention the binding pH value is of between about 1.0 to about 12.0, preferably about 1, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, about 12, more preferably about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9.

The immunoglobulin preparation is characterized not only by its pH value but also by its “conductivity”, i.e., its ability of conduct an electrical current which is a direct measure of the content of salt of the preparation. In fact, the terms “ionic strength”, salt content” and “conductivity” are all measures of the same parameter: the salt content of the immunoglobulin preparation. The salt content will depend on the solution buffer used, specifically on the ions contained therein.

Conductivity is measured with a probe and a meter. Voltage is applied between two electrodes in a probe immersed in the sample water. The drop in voltage caused by the resistance of the water is used to calculate the conductivity per centimeter. All of the current chromatography equipment's have a conductivity measurement to determine the conductivity of the solution present in the columns of chromatography. In a particular embodiment the conductivity of the preparation in step (i) is of between about 5.0 miliSiemens per centimeter (mS/cm) and about 15.0 mS/cm, preferably about 5 mS/cm, about 5.5 mS/cm, about 6 mS/cm, about 6.5 mS/cm, about 7 mS/cm, about 7.5 mS/cm, about 8 mS/cm, about 8.5 mS/cm, about 9 mS/cm, about 9.5 mS/cm, about 10 mS/cm, about 10.5 mS/cm, about 11 mS/cm, about 11.5 mS/cm, about 12 mS/cm, about 12.5 mS/cm, about 13 mS/cm, about 13.5 mS/cm, about 14 mS/cm, about 14.5 mS/cm, about 15 mS/cm. In a more particular embodiment, the conductivity of the preparation in step (i) is of about 10.0 mS/cm.

In addition to the pH value and the conductivity, the immunoglobulin preparation is further characterized by the concentration of immunoglobulins in the preparation. The concentration of the immunoglobulins in the preparation can be measured by several methods well known to the expert in the field, such as measuring the UV absorbance of the preparation at 280 nm, the Bradford assay and the BCA assay, which is a colorimetric assay that utilizes the reduction of Cu2+ ions by proteins, and subsequent binding of BCA (Copeland R. A., 1994, Methods for Protein Quantitation. In: Methods for Protein Analysis. Springer, Boston, MA). In a particular embodiment of the method of the invention the immunoglobulin concentration in the immunoglobulin preparation is step (i) is of between about 0.1 g/L to about 100 g/L, preferably about 1 g/L, about 10 g/L, about 20 g/L, about 30 g/L, about 40 g/L, about 50 g/L, about 60 g/L, about 70 g/L, about 80 g/L, about 90 g/L, more preferably about 1.5 g/L, about 0.5 g/L, about 1 g/L, about 1.5 g/L, about 2 g/L, about 2.5 g/L, about 3 g/L, about 3.5 g/L, about 4 g/L, about 4.5 g/L, about 5 g/L, about 6 g/L, about 7 g/L, about 8 g/L, about 9 g/L, about 10 g/L, about 11 g/L, about 12 g/L, about 13 g/L, about 14 g/L, about 15 g/L, about 16 g/L, about 17 g/L, about 18 g/L, about 19 g/L, about 20 g/L, about 21 g/L, about 22 g/L, about 23 g/L, about 24 g/L, about 25 g/L, about 26 g/L, about 27 g/L, about 28 g/L, about 29 g/L.

### Method of the Invention—Second Step

Once the preparation of the step (i) of the invention has the correct pH in order to net charge the immunoglobulins, the next step is contacting said preparation with a multimodal ion exchanger. The term “multimodal ion exchanger” as used herein refers to a component of the solid phase which is suited for use in multimodal chromatography (MMC), and which has multiple modes of interaction with the protein(s) of interest. These interactions may be introduced via a chemical scaffold that links the new interaction with the primary one, resulting in a well-defined three-dimensional new multimodal ligand or it can be achieved by presenting different interaction groups which are independently attached to the matrix. The interactions that can be available are quite diverse, for example, electrostatic, hydrophobic, TT-TT, hydrogen bonding, and thiophilic interactions. The terms “multimodal ion exchanger”, “mixed-mode ion exchanger”, “multimodal ion exchange ligand” and “mixed-mode ion exchange ligand” are used herein as synonyms.

According to step (ii) of the present invention, the MME comprises a ligand having a hydrophobic moiety and a moiety which is charged. In a particular embodiment of the method of the invention, the multimodal ion exchanger ligand further comprises one moiety selected from an electrostatic moiety, a (different) hydrophobic moiety, an ion exchanger moiety, and hydrogen bonding moiety and a thiophilic moiety.

As stated, MMC takes advantage of several modes of binding with proteins which allows the tailoring of specific ligands. The most common multimodal ligands comprise ion exchange, hydrogen bonding and hydrophobic interaction groups which interact with the target protein.

The term “ion exchanger moiety” refers to a negatively charged solid phase (i.e., cation exchange group) or a positively charged solid phase (i.e., anionic exchange group). In a particular embodiment the multimodal ion exchanger is a multimodal anion exchanger or a multimodal cationic exchanger. In a particular embodiment of the method the multimodal ion exchanger comprises an anion exchange moiety selected from quaternary ammonium group, diethylaminoethane, diethylaminopropyl, dietil-2-hidroxipropilaminoetilo (QAE), trietilaminoetilo (TEAE), trimetilaminoetilo and ammine group. In a particular embodiment of the method the multimodal ion exchanger comprises a cation exchange moiety selected from a group consisting of: Sulphonic acid, Sulfopropyl (SP), Methyl sulfonate(S) and Carboxymethyl (CM).

The term “hydrophobic moiety” as used herein refers to chemical groups such as amino acid side chains which are substantially water insoluble, but soluble in an oil phase, with the solubility in the oil phase being higher than that in water or in an aqueous phase. Hydrophobic moieties when in close proximity interact with one another in order to form a non-aqueous environment, forming a hydrophobic interaction. Hydrophobic moieties in an ion multimodal exchanger are typically given by aliphatic or aromatic groups. In a particular embodiment of the method of the invention the multimodal ion exchanger comprises a hydrophobic moiety selected from an aliphatic group, an aromatic group or a combination of both.

In a particular embodiment of the method of the invention the multimodal ion exchanger comprises an aliphatic group selected from butyl or octyl.

In another particular embodiment of the method of the invention the multimodal ion exchanger comprises an aliphatic group selected from a group consisting of: Alkane, Alkyne, Alkene, Diene, Cycloalkene, Cycloalkane, Cyclohexane, Octane, Alkane, Terpene and Polyene.

In another particular embodiment of the method of the invention the multimodal ion exchanger comprises an aromatic group selected from the group consisting of: benzene, phenyl, toluene, ethylbenzene, p-xylene, m-xylene, mesitylene, 2-phenylhexane, biphenyl, phenol, nitrobenzene and benzoic acid.

In a particular embodiment of the method of the invention the multimodal ion exchanger comprises phenyl as an aromatic group.

The term “hydrogen bonding moiety” as used herein refers to a group which can serve as donors or acceptors of a hydrogen bond, a primarily electrostatic force of attraction between a hydrogen (H) atom which is covalently bound to a more electronegative atom or group, and another electronegative atom bearing a lone pair of electrons—the hydrogen bond acceptor (Ac). The most frequent donor and acceptor atoms are nitrogen (N), oxygen (O), and Sulfur(S)

In a particular embodiment of the method of the invention the multimodal ion exchanger comprises a hydrogen bonding group selected from nitrogen, oxygen and fluorine.

The multimodal ion exchanger of the method of the invention may be bound to a solid substrate which provides a physical scaffold for the presentation of the multimodal ion exchanger to the immunoglobulin preparation, termed the “matrix”. Matrices have ideally low level of nonspecific interaction or are inert.

In a particular embodiment of the method of the invention the multimodal ion exchanger comprises a matrix. In another particular embodiment of the method of the invention the matrix is selected from a group consisting of: Polystyrene, agarose 4%, Agarose 6% with dextran chains coupled to agarose, cross-linked agarose beads, silica, cross-linked hydroxylated methacrylic polymers, cellulose, dextran, polyacrylamide and polystyrene. In another particular embodiment of the method of the invention the matrix is preferably high-flow agarose. In a more particular embodiment of the method of the invention the matrix is cross-linked high-flow agarose. Methods and processes to cross-link agarose beads are widely known in the field and can be found in the following documents: EP0153910A1, U.S. Pat. No. 3,860,573A, and WO2019192059A1.

Matrices can have different shapes and/or arrangements. In a particular embodiment of the method of the invention the matrix is provided as a resin, wherein said resin has a median particle size of the cumulative volume distribution of about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, preferably 40 μm.

Normally the most common way to secure the multimodal ion exchanger to the matrix is to couple both with a covalent bond. This can be accomplished through several immobilization chemistries such as coupling through amino groups, through NHS ester activated-matrices and aldehyde-activated matrices.

In order to obtain the best possible yield from the multimodal chromatography the use of a chemical spacer between the solid substrate and the multimodal ion exchanger may be required. The term “chemical spacer” as used herein describes a chemical moiety that is covalently attached to a chemical residue of the solid substrate and/or the multimodal ion exchanger and/or interposed between the two. In another particular embodiment of the method of the invention the multimodal ion exchanger is attached to the solid substrate by a chemical spacer, wherein the chemical spacer is aminocaproic acid.

In a particular embodiment of the method of the invention the multimodal anion exchanger is selected from a group consisting of: Alkylamine, α,ω-Diamino alkane, Phenylalkylamine, 2-Amino-1-phenyl-1,3-propanediol, N-Benzyl-N-methyl ethanol amine, 4-Mercaptoethylpyridine (4-MEP), 2-Aminomethylpyridine, Mercaptomethylimidazole, 2-Mercaptobenzimidazole, Tryptamine, 5-Aminoindole, tryptophan, phenylpropylamine, hexylamine, N,N-dimethyl-2-phenoxyethan-1-amine, 4-amino-4,6-dideoxy-beta-D-glucopyranose, Hydroxylapatite and any combination thereof. In a particular embodiment of the method of the invention, the multimodal anion exchanger is N-benzyl-N-methyl ethanolamine group.

In a particular embodiment of the method of the invention the multimodal cation exchanger is selected from a group consisting of: Sulphonic acid, Sulfopropyl (SP), Methyl sulfonate(S) and Carboxymethyl (CM).

In a particular embodiment the combination of a matrix and a multimodal anion exchanger results in a resin wherein said resin is selected from the group consisting of: tryptophan to bound cross-linked hydroxylated methacrylic polymers, 4-Mercaptoethylpyridine (4-MEP) bound to cellulose, phenylpropylamine bound to cellulose, hexylamine bound to cellulose, N-benzyl-N-methyl ethanolamine bound to cross-linked agarose beads, wherein the bonds between the MME and the matrix are covalent bonds.

The second step of the method of the invention can be described as several sub-steps, which entail the equilibration of the multimodal ion exchanger, the loading of the multimodal ion exchanger with the immunoglobulin preparation of step (i) of the method of the invention and the washing of the loaded multimodal ion exchanger.

Therefore, in a particular embodiment of the method of the invention wherein the adjusting step (i) is carried so that the binding pH value provides a positive net charge to the immunoglobulin molecules and wherein the contacting step (ii) comprises the steps of:
(i) Equilibrating the multimodal anion exchanger with a loading buffer by allowing the loading buffer to flow through the multimodal anion exchanger wherein the pH of the loading buffer is of between about 1.0 to 12.0 and has a conductivity of between about 0.1 mS/cm to about 30 mS/cm;
(ii) Loading the multimodal anion exchanger with the immunoglobulin preparation by allowing the immunoglobulin preparation to flow through the multimodal anion exchanger wherein the concentration of immunoglobulin is of less than about 100 g/L, and, optionally,
(iii) Washing the multimodal anion exchanger by allowing a washing buffer to flow through the multimodal anion exchanger wherein the washing buffer has a pH of between about pH 1.0 to about pH 12.0 and a conductivity of between about 0.1 mS/cm to about 30 mS/cm.

The terms “buffer” or “buffered solution” are used interchangeable and as used herein refer to an aqueous formulation comprising a chemical compound employed for the purpose of stabilizing the pH of an aqueous solution within a specified range. Phosphate is one example of a buffering compound. Other common examples include but are not limited to compounds such as acetate, citrate, borate, MES, Tris, and HEPES, phosphate buffered saline (PBS) among many others.

The term “loading buffer” as used herein refers to a buffer required to establish a specified set of conditions to mediate the bringing into contact of the multimodal ion exchanger with the immunoglobulin preparation. The term “washing buffer” refers to a buffer formulated to displace unbound contaminants from the multimodal ion exchanger. “Elution buffer” refers to a buffer formulated to displace the one or more components bound to the multimodal ion exchanger. The term “buffered solution” as used herein refers to and includes the terms “loading buffer”, “wash buffer” and “elution buffer”.

In a particular embodiment of the subset (i) of the contacting step (ii) of the method of the invention, the loading buffer is the buffered solution.

As previously mentioned, one of the main purposes of a buffer solution is to maintain a stable pH. In another particular embodiment of the subset (i) of the contacting step (ii) of the method of the invention the pH of the loading buffer is of about 1.0, about 2.0, about 3.0, about 4.0, about 5.0, about 7.0, about 8.0, about 9.0, about 10.0, about 11.0, about 12.0. Likewise, the buffer solution, i.e., the salt content of said buffer solution, affects the conductivity of said solution. In a particular embodiment of the subset (i) of the contacting step (ii) of the method of the invention, the conductivity of the loading buffer is of about 1 mS/cm, about 2 mS/cm, about 3 mS/cm, about 4 mS/cm, about 5 mS/cm, about 6 mS/cm, about 7 mS/cm, about 8 mS/cm, about 9 mS/cm, about 10 mS/cm, about 11 mS/cm, about 12 mS/cm, about 13 mS/cm, about 14 mS/cm, about 15 mS/cm, about 16 mS/cm, about 17 mS/cm, about 18 mS/cm, about 19 mS/cm, about 20 mS/cm, about 21 mS/cm, about 22 mS/cm, about 23 mS/cm, about 24 mS/cm, about 25 mS/cm, about 26 mS/cm, about 27 mS/cm, about 28 mS/cm, about 29 mS/cm, about 30 mS/cm.

Once the multimodal ion exchanger is equilibrated, the immunoglobulin preparation can be brought into contact with the multimodal ion exchanger, i.e., the “loading of the multimodal anion exchanger with the immunoglobulin preparation”. This process is achieved by allowing the immunoglobulin preparation to flow through the multimodal anion exchanger, a process during which the immunoglobulins will bound to the multimodal ion exchanger. In a particular embodiment of the subset (ii) of the contacting step (ii) of the method of the invention, the concentration of the immunoglobulin bound to the multimodal exchanger is of about 1 g/L, about 10 g/L, about g/L, about 30 g/L, about 40 g/L, about 50 g/L, about 60 g/L, about 70 g/L, about 80 g/L, about 90 g/L, about 100 g/L.

Once the loading is complete, the next step is to clear contaminants that are unbound to the multimodal ion exchanger from the solution surrounding the multimodal ion exchanger. This is achieved by flowing through the multimodal ion exchanger a washing buffer. In a particular embodiment of the subset (iii) of the contacting step (ii) of the method of the invention, the washing buffer is the buffered solution.

In another particular embodiment of the subset (iii) of the contacting step (ii) of the method of the invention the pH of the washing buffer is about 1, about 1, about 2, about 3, about 4, about 5, about 6, about 7, about 8, about 9, about 10, about 11, about 12.

In yet another particular embodiment of the subset (iii) of the contacting step (ii) of the method of the invention, the conductivity of the washing buffer is of about about 1 mS/cm, about 2 mS/cm, about 3 mS/cm, about 4 mS/cm, about 5 mS/cm, about 6 mS/cm, about 7 mS/cm, about 8 mS/cm, about 9 mS/cm, about 10 mS/cm, about 11 mS/cm, about 12 mS/cm, about 13 mS/cm, about 14 mS/cm, about 15 mS/cm, about 16 mS/cm, about 17 mS/cm, about 18 mS/cm, about 19 mS/cm, about 20 mS/cm, about 21 mS/cm, about 22 mS/cm, about 23 mS/cm, about 24 mS/cm, about 25 mS/cm, about 26 mS/cm, about 27 mS/cm, about 28 mS/cm, about 29 mS/cm, about 30 mS/cm.

In a particular embodiment of the method of the invention the contacting step (ii) and the elution step (iii) (see below) are carried out in a buffered solution. In another particular embodiment of the method of the invention, the contacting step (ii) and the elution step (iii) are carried out in the same buffered solution. In another particular embodiment of the method of the invention the buffered solution is selected from a group consisting of: acetate buffer, citrate buffer, borate buffer, 2-ethanesulfonic acid (MES) buffer, trisaminomethane (Tris) buffer, 4-(2-hydroxyethyl)-1-piperazineethanesulfonic acid (HEPES) buffer, phosphate buffered saline (PBS) buffer or any combination thereof. In a more particular embodiment, the buffered solution is an acetate buffer at about 10 mM, about 20 mM, about 30 mM, about 40 mM, about 50 mM, about 60 mM, about 70 mM, about 80 mM, about 90 mM, about 100 mM concentration, preferably at 50 mM concentration.

In one more particular embodiment of the contacting step (ii) of the method of the invention the loading buffer and/or the washing buffer is allowed to flow through the multimodal anion exchanger at a rate of at least 50 cm/h, at least 100 cm/h, at least 150 cm/h, at least 200 cm/h, at least 250 cm/h, at least 300 cm/h, at least 350 cm/h, at least 400 cm/h, at least 450 cm/h, at least 500 cm/h, at least 550 cm/h, at least 600 cm/h.

The term “residence time” in the context of the present description refers to the total time a fluid parcel, in the present case a parcel of a buffer or the washing buffer, has spent inside a control volume, in the present case the volume of multimodal ion exchanger used in the experiment. The residence time can be calculated by the following formula: residence time (min)=[multimodal ion exchanger height (cm)/flow through velocity/rate (cm/h)]×60.

In one more particular embodiment of the contacting step (ii) of the method of the invention the loading buffer and/or the washing buffer is allowed to flow through the multimodal anion exchanger with a residence time of at least 1 min, at least 2 min, at least 3 min, at least 4 min, at least 5 min, at least 6 min, at least 7 min, at least 8 min, at least 9 min, at least 10 min, at least 11 min, at least 12 min, at least 13 min, at least 14 min, at least 15 min, at least 20 min, at least 30 min,

In a more particular embodiment of the contacting step (ii) of the method of the invention the loading buffer and/or the washing buffer is allowed to flow through the multimodal anion exchanger with a residence time of at least 1 min.

### Method of the Invention—Third Step

In order to obtain a final purified immunoglobulin preparation, the immunoglobulins bound to the multimodal ion exchanger have to be released and recovered. After loading all the sample into the multimodal ion exchanger and washing it to remove all the nonbinding proteins, the buffer conditions are altered in order to elute the bound proteins. The term “eluting” refers to the process of extracting said immunoglobulins absorbed to the multimodal ion exchanger. Most frequently, proteins are eluted by increasing the ionic strength (salt concentration) of the buffer or, occasionally, by changing the pH. As ionic strength increases, the salt ions (typically Na+ or Cl—) compete with the bound components for charges on the surface of the medium and one or more of the bound species begin to elute and move down the column. The proteins with the lowest net charge at the selected pH will be the first ones eluted from the column as ionic strength increases. Similarly, the proteins with the highest charge at a certain pH will be most strongly retained and will be eluted last. The higher the net charge of the protein, the higher the ionic strength that is needed for elution. By controlling changes in ionic strength using different forms of gradient, proteins are eluted differently in a purified, concentrated form.

In one embodiment of the present invention the elution is carried out by decreasing the pH in a constant manner, i.e., by performing a pH gradient. In a particular embodiment the pH gradient during the elution step (iii) of the method of the invention has a starting pH of about 8.0 and an end pH of about 2.0, a starting pH of about 7.5 and an end pH of about 2.5, a starting pH of about 7.0 and an end pH of about 3.0, a starting pH of about 6.5 and an end pH of about 3.0, a starting pH of about 6.0 and an end pH of about 3.5, a starting pH of about 5.5 and an end pH of about 4.0, a starting pH of about 5.0 and an end pH of about 4.5. In a more particular embodiment, the pH gradient during the elution step (iii) of the method of the invention has a starting pH of about 6.0, and an end pH of about 4.0.

In another embodiment of the present invention the elution is carried out by increasing the pH in a constant manner, i.e., by performing a pH gradient. In a particular embodiment the pH gradient during the elution step (iii) of the method of the invention has a ending pH of about 8.0 and a start pH of about 2.0, a ending pH of about 7.5 and an start pH of about 2.5, a ending pH of about 7.0 and an start pH of about 3.0, a ending pH of about 6.5 and an start pH of about 3.0, a ending pH of about 6.0 and an start pH of about 3.5, a ending pH of about 5.5 and an start pH of about 4.0, a ending pH of about 5.0 and an start pH of about 4.5. In a more particular embodiment, the pH gradient during the elution step (iii) of the method of the invention has a ending pH of about 6.0, and an start pH of about 4.0.

In the present invention, the step (iii) of the method of the invention the elution is done with a decreasing pH gradient at constant conductivity. In the present invention, the step (iii) of the method of the invention the elution is done with a increasing pH gradient at constant conductivity. In a particular embodiment of the method of the invention the conductivity during the elution step (iii) is maintained at a constant value of about 1 mS/cm, about 2 mS/cm, about 3 mS/cm, about 4 mS/cm, about 5 mS/cm, about 6 mS/cm, about 7 mS/cm, about 8 mS/cm, about 9 mS/cm, about 10 mS/cm, about 11 mS/cm, about 12 mS/cm, about 13 mS/cm, about 14 mS/cm, about 15 mS/cm, about 16 mS/cm, about 17 mS/cm, about 18 mS/cm, about 19 mS/cm, about 20 mS/cm, about 21 mS/cm, about 22 mS/cm, about 23 mS/cm, about 24 mS/cm, about 25 mS/cm, about 26 mS/cm, about 27 mS/cm, about 28 mS/cm, about 29 mS/cm, about 30 mS/cm. In a particular embodiment of the method of the invention the conductivity during the elution step (iii) is maintained at a constant value of about 10 mS/cm

The objective of the elution is to obtain a preparation containing immunoglobulins, which are purified in relation to the initial immunoglobulin preparation. The term “purified” as used herein refers to the process of removing or reducing contaminants present in the preparation together with the immunoglobulins. Examples of said contaminants are residual “host cell proteins (HCP)”, nucleic acids such as DNA and RNA, insulin and leached protein A. The term “host cell proteins” or “HCP” in the present context refers to process-related protein impurities that are produced by the host organism during manufacturing and production of the immunoglobulins. In a particular embodiment the final purified immunoglobulin preparation obtained in step (iii) of the method of the invention is characterized by having a reduction of at least 70%, at least 80%, at least 90%, at least 91%, at least 92%, at least 93%, at least 94%, at least 95%, at least 96%, at least 97%, at least 98%, at least 99%, at least 99%, at least 99.1%, at least 99.2%, at least 99.3%, at least 99.4%, at least 99.5%, at least 99.6%, at least 99.7%, at least 99.8%, at least 99.9% contaminants in comparison to the immunoglobulin preparation of step (i) of the method of the invention.

### Method of the Invention—Steps Performed Before the Method of the Invention

Protein purification strategies may include further methods and processes that improve, facilitate and/or increase parameters related to the process and or protein, e.g., protein yield, purification level, process efficiency, etc.

In a particular embodiment the method of the invention further comprises a step of viral inactivation of the immunoglobulin preparation at low pH prior to step (i).

The term “viral inactivation” refers to rendering a virus contained in the mixture nonfunctional. The virus may originate from the source of immunoglobulin/protein production, downstream processing steps or manufacturing conditions. Methods of rendering a virus nonfunctional or removing a virus include heat activation, pH inactivation, chemical inactivating agents, etc. The term “pH viral inactivation” includes subjecting a virus to a pH sufficient to render the virus nonfunctional, e.g. a pH between about 2.0 and 8.0. In a particular embodiment the method of the invention further comprises a step of viral inactivation wherein the viral inactivation at low pH comprises bringing the immunoglobulin preparation to a pH value between about 2.0 and about 4.0, preferably about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9.

In a particular embodiment the method of the invention further comprises a step of viral inactivation wherein said step comprises the subsets of:
(i) Adjusting the pH of the immunoglobulin preparation to between about 4.0 and about 2.0, preferably 3.6;
(ii) Incubate with stirring for between 15 min and 100 min, preferably for 60 min;
(iii) Adjusting the pH to at least 6.0 and adjust conductivity to at least 3 ms/cm; and
(iv) Filtering the solution through single use depth filters.

The term “depth filters” as used herein refers to filters that use a porous filtration medium to retain particles throughout the medium, rather than just on the surface of the medium. These filters are commonly used when the fluid to be filtered contains a high load of particles because, relative to other types of filters, they can retain a large mass of particles before becoming clogged.

In another particular embodiment the method of the invention further comprises a step prior to step (i) whereby the immunoglobulin preparation is enriched in immunoglobulin molecules by affinity chromatography with an immobilized immunoglobulin specific ligand.

The term “affinity chromatography” or “affinity purification” refers to a separation method based on a specific binding interaction between a ligand immobilized or coupled to a solid support and its binding partner. When a complex mixture is passed over the column, those molecules having specific binding affinity to the ligand become bound. After other sample components are washed away, the bound molecule is stripped form the support, resulting in its purification from the original mixture. Each specific affinity system requires its own set of conditions known to a person of ordinary skill in the art.

The term “ligand”, “affinity ligand” or “specific ligand” refers to metals (e.g., Cd+2, Co+2, Cu+2, Ga+3, Fe+3, Ni+2, and Zn+2), dyes (e.g., Cibacron Blue and variants thereof), glutathione, subtilisin, Protein A, Protein G, Protein A/G, Protein L, boronate, avidin, streptavidin, biotin, anti-c-Myc, anti-HA, nucleotides, coenzymes, antibodies, heparin, antigens (especially for antibodies with a known specificity), and other known affinity ligands. In a particular embodiment the method of the invention further comprises a step prior to step (i) whereby the immunoglobulin preparation is enriched in immunoglobulin molecules by affinity chromatography with an immobilized immunoglobulin specific ligand, wherein the specific immunoglobulin specific ligand is protein A.

In a particular embodiment of the method of the invention, the solution obtained after the affinity chromatography step is characterized by having a pH at between about 2.0 to about 8.0, preferably at between about 4.0 to about 5.0.

Methods of affinity chromatography normally include steps of equilibrating the ligand resin, loading the ligand resin with the solution containing the protein of interest, washing contaminants present in said solution and eluting the protein of interest bound to the ligand. The expert in the field will be aware of the ideal parameters and best procedures to perform the affinity chromatography process in order to obtain

In a particular embodiment of the method of the invention the solution obtained after the affinity chromatography step is characterized by having a conductivity of between about 1.0 ms/cm to about 30.0 ms/cm, preferably between about 2.0 ms/mc to about 20.0 ms/mc, more preferably between about 3.0 ms/mc to about 10.0 ms/mc. In a particular embodiment of the method of the invention, the solution obtained after the affinity chromatography step is characterized by having an immunoglobulin concentration of between about 1.0 g/L to about 50 g/L, preferably of between about 20.0 g/L to about 25.0 g/L.

In a particular embodiment of the method of the invention the solution obtained after the affinity chromatography step is characterized by having a pH at between about 2.0 to about 5.0, a conductivity of between about 1.0 ms/mc to about 30.0 ms/mc and an immunoglobulin concentration of between about 1.0 g/L to about 50.0 g/L.

In a particular embodiment of the method of the invention further comprises a step whereby the immunoglobulin preparation is enriched in immunoglobulin with an immunoglobulin specific ligand and the step of viral inactivation is carried out after the step of immunoglobulin enrichment by affinity chromatography.

Method of the Invention—Steps after the Method of the Invention

Following the purification of the immunoglobulins by the method of the invention further processes may be required to bring the purified immunoglobulin preparation into a form suitable for therapeutic administration, for example, to a non-human animal or to a human being. Such further processing may include any combination of ultrafiltration, nanofiltration, concentration, and diafiltration of the purified preparation of the protein of interest. Ultrafiltration is a process for concentrating the preparation of the protein of interest. Proteins are filtered from other molecules in solution based on the membrane pore size or molecular weight cutoff. Ultrafiltration is a membrane liquid-separation technology, which has high rejection of virtually all dissolved solutes, such as multivalent ions like calcium, and low rejection of monovalent ions such as chloride. Diafiltration is used to exchange the protein of interest into a desired buffer (e.g., from an elution buffer into a stable formulation buffer). Ultrafiltration and diafiltration typically employ tangential flow filtration. Ultrafiltration and diafiltration filters are characterized by their molecular weight “cut-off”, a term which as used herein refers to the smallest molecular-weight species for which the membrane displays more than 90% rejection.

In a particular embodiment, the method of the invention further comprises a step of nanofiltration performed after the elution step (iii) wherein the nanofiltration is carried out using a filter having a mean pore size of about 20 nm and wherein the eluate is recovered.

In a particular embodiment of the method of the invention the eluate obtained after the nanofiltration is characterized by having a pH of between about 2.0 and 8.0, preferably between about 4.0 to about 6.0, more preferably between about 5.0 to about 5.8. In a particular embodiment of the method of the invention the eluate obtained after the nanofiltration is characterized by having a conductivity of between about 10.5 ms/mc to about 1000 ms/mc, preferably between 5 ms/mc to about 50 ms/mc, preferably between 50 ms/mc to about 100 ms/mc, preferably between about 200 ms/mc to about 900 ms/mc, preferably between about 300 ms/mc to about 800 ms/mc, preferably between 400 ms/mc and 600 ms/mc. In a particular embodiment of the method of the invention the eluate obtained after the nanofiltration is characterized by having an immunoglobulin concentration of between about 0.1 g/L to about 100.0 g/L, between about 1.0 g/L, about 10.0 g/L to about 40.0 g/L, preferably about 10 g/L, about 11 g/L, about 12 g/L, about 13 g/L, about 14 g/L, about 15 g/L, about 16 g/L, about 17 g/L, about 18 g/L, about 19 g/L, about 20 g/L, about 21 g/L, about 22 g/L, about 23 g/L, about 24 g/L, about 25 g/L, about 26 g/L, about 27 g/L, about 28 g/L, about 29 g/L, about 30 g/L, about 31 g/L, about 32 g/L, about 33 g/L, about 34 g/L, about 35 g/L, about 36 g/L, about 37 g/L, about 38 g/L, about 39 g/L, about 40 g/L, about 50 g/L, about 60 g/L, about 70 g/L, about 80 g/L, about 90 g/L, about 100 g/L.

In a particular embodiment of the method of the invention the eluate obtained after the nanofiltration is characterized by having a pH of between about 1.0 to about 12.0, a conductivity of between about 0.5 ms/mc to about 1000 ms/mc, and an immunoglobulin concentration of between about 1.0 g/L to about 100.0 g/L.

In a particular embodiment, the method of the invention further comprises a step of ultrafiltration/diafiltration after the elution step (iii), wherein the ultrafiltration/diafiltration is carried out using a filter having a cut-off of 30 kDa wherein the recovered fraction is the retentate.

The term “recovered fraction” as used herein refers to the material which is kept for further processing after the ultrafiltration/diafiltration. The term “retentate” as used herein refers to the material retained by the membrane.

In a particular embodiment of the method of the invention the retentate obtained after the ultrafiltration/diafiltration is characterized by having a pH of between about 2.0 and 8.0, preferably between about 4.0 to about 6.0, more preferably between about 5.0 to about 5.8. In a particular embodiment of the method of the invention the retentate obtained after the ultrafiltration/diafiltration is characterized by having a conductivity of between about 0.5 s/mc to about 1000 ms/mc, preferably between about 1 ms/mc and about 100 ms/mc, about 5 ms/mc and about 10 ms/mc, preferably between about 200 ms/mc to about 900 ms/mc, preferably between about 300 ms/mc to about 800 ms/mc, preferably between 400 ms/mc and 600 ms/mc. In a particular embodiment of the method of the invention the retentate obtained after the ultrafiltration/diafiltration is characterized by having an immunoglobulin concentration of between about 1.0 g/L to about 100.0 g/L, between about 10.0 g/L to about 40.0 g/L, preferably about 10 g/L, about 11 g/L, about 12 g/L, about 13 g/L, about 14 g/L, about 15 g/L, about 16 g/L, about 17 g/L, about 18 g/L, about 19 g/L, about 20 g/L, about 21 g/L, about 22 g/L, about 23 g/L, about 24 g/L, about 25 g/L, about 26 g/L, about 27 g/L, about 28 g/L, about 29 g/L, about 30 g/L, about 31 g/L, about 32 g/L, about 33 g/L, about 34 g/L, about 35 g/L, about 36 g/L, about 37 g/L, about 38 g/L, about 39 g/L, about 40 g/L, about 50 g/L, about 60 g/L, about 70 g/L, about 80 g/L, about 90 g/L, about 100 g/L.

In a particular embodiment of the method of the invention the retentate obtained after the ultrafiltration/diafiltration is characterized by having a pH of between about 1.0 to about 12.0, a conductivity of between about 0.5 ms/mc to about 1000 ms/mc, and an immunoglobulin concentration of between about 1.0 g/L to about 100.0 g/L.

In another particular embodiment of the method of the invention, the step of ultrafiltration/diafiltration is carried out after the step of nanofiltration.

The method of the invention allows for the purification of proteins, preferably immunoglobulins. In a particular embodiment, the method of the invention further comprises formulating the immunoglobulin under conditions adequate for storage.

The expert in the field will be aware of the best ways to store the eluted immunoglobulins.

### Products of the Invention

The method of the invention is designed to obtain proteins at a high level of purification allowing the modulation of charge variants as well as removal of aggregates, being specifically adapted for the purification of immunoglobulins. Therefore, an aspect of the present invention relates to an immunoglobulin obtained by the method of the invention, from here onwards the immunoglobulin of the invention.

The term “immunoglobulin” has been previously defined and such definition and relates embodiments are equally valid for the current aspect.

Another aspect of the present invention relates to a pharmaceutical composition, from here onwards the pharmaceutical composition of the invention, comprising the immunoglobulin of the invention or an immunoglobulin obtained by the method of the invention and an acceptable adjuvant.

The term “acceptable adjuvant carrier” as used herein refers to a vehicle, diluent or expedient that is administered with the active ingredient. Such pharmaceutical excipients can be sterile liquids, such as water and oils, including those of petroleum, animal, vegetable or synthetic origin, such as peanut oil, soybean oil, mineral oil, sesame oil and similar. Water or saline aqueous solutions and aqueous dextrose and glycerol solutions are preferably used as vehicles. Suitable pharmaceutically acceptable vehicles include, for example, water, salt solutions, alcohol, vegetable oils, polyethylene glycols, gelatin, lactose, amylose, magnesium stearate, talc, surfactants, silicic acid, viscous paraffin, perfume oil, monoglycerides and diglycerides of fatty acids, fatty acid esters petroetrals, hydroxymethyl cellulose, polyvinylpyrrolidone and similars. In addition, the term “acceptable adjuvant carrier” also includes any other pharmaceutical active agent(s) or drug(s), such as a chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. When the pharmaceutical composition is formulated as an injectable solution, the pharmaceutical acceptable carrier may include any isotonic carrier such as, without limitation, normal saline (about 0.90% w/v of NaCl in water, about 300 mOsm/L NaCl in water, or about 9.0 g NaCl per liter of water), about 5% dextrose in water, or Ringer's lactate.

In a particular embodiment, the pharmaceutical composition of the invention comprises a therapeutically effective amount of the immunoglobulin of the invention or of an immunoglobulin obtained by the method of the invention.

The expression “therapeutically effective amount” as used herein defines an amount that can be administered or applied to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

### Uses of the Invention

All previous definitions and embodiments related to previous aspects are also applicable to the current aspects and their embodiments.

Another aspect of the invention relates to the immunoglobulin of the invention, or an immunoglobulin obtained by the method of the invention for use in medicine.

Another further aspect relates to the immunoglobulin of the invention, or an immunoglobulin obtained by the method of the invention for use in the treatment of cancer, from here onwards the treatment use of the invention.

The term “treatment” as used herein refers to an intervention (e.g., the administration of the immunoglobulin of the invention) which cures, ameliorates or lessens the symptoms of a disease or removes (or lessens the impact of) its cause(s) (for example, the eradication of cancer cells from the subject). In this case, the term is used synonymously with the term “therapy”. In the context of the present invention the immunoglobulin of the invention or an immunoglobulin obtained by the method of the invention for use in the treatment of cancer is used in a therapeutically effective amount.

In a particular embodiment of the treatment use of the invention the cancer is selected from a group consisting of: acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vagina, cancer of the vulva, cholangiocarcinoma, chronic lymphocytic leukemia, chronic myeloid cancer, colon cancer, esophageal cancer, uterine cervical cancer, gastrointestinal carcinoid tumor, glioma, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, cancer of the oropharynx, ovarian cancer, cancer of the penis, pancreatic cancer, peritoneum, omentum, and mesentery cancer, pharynx cancer, prostate cancer, rectal cancer, renal cancer, skin cancer, small intestine cancer, soft tissue cancer, stomach cancer, testicular cancer, thyroid cancer, cancer of the uterus, ureter cancer, urinary bladder cancer, solid tumors, and liquid tumors.

The method of the invention finds also use in reducing the level of undesirable contaminants, aggregates and impurities from protein solutions.

Therefore, another aspect of the present invention relates to the use of the method of the invention to reduce the level of high molecular weight aggregates in the immunoglobulin preparation.

One more aspect of the present invention relates to the use of the method of the invention to reduce the contents of charge variants in the immunoglobulin preparation.

The term “charge variants” as used herein refers to multiple forms or fractions of the immunoglobulin in the preparation, which differ in the charge, molecular weight due to post-translation modifications for example, and other properties. Several methods can be used to determine the charge variants of immunoglobulins. Examples of such techniques are capillary zone electrophoresis (CZE) and ion exchange chromatography (IEX) analysis. Capillary zone electrophoresis (CZE) is an analytical technique that allows the analysis of ionized or ionizable compounds. Analytes are simply separated according to their charge/hydrodynamic ratio and migrate towards anode or cathode according to their charges. CZE does not require denaturants or solid-phase interfaces, permitting the inherent heterogeneity of mAbs to be analyzed close to their native state. During a CZE analysis, the analyte apparent mobility is directly related to its migration in the capillary. The apparent mobility of an analyte is a vector sum of the electrophoretic mobility of the analyte plus the electroosmotic mobility of the buffer. The separation is based on the differences is electrophoretic mobility, which is directed proportional to the charge on the molecule, and inversely proportional to the viscosity of the solvent and radius of the atom.

Ion exchange chromatography is a technique for separating compounds based on their net charge. Ion exchange chromatography media contain negatively or positively charged functional groups covalently bound to a solid support, yielding either a cation or anion exchanger, respectively. Charged compounds are absorbed and retained by an ion exchanger having the opposite charge, whereas compounds that are neutral or have the same charge as the media pass through the void volume and are eluted from the column. The binding of the charged compounds is reversible, and adsorbed compounds are commonly eluted with a salt or pH gradient. Ion exchange media are available in various particle sizes, ionic forms, and purity ranges.

One more aspect of the present invention relates to the use of the method of the invention to reduce the contents of impurities in the immunoglobulin preparation.

In a particular embodiment of the use of the method of the invention to reduce the contents of impurities in the immunoglobulin preparation, the impurities are selected from a group consisting of: nucleic acids, DNA, RNA, host cell proteins (HCPs), insulin, protein A, and any combination thereof.

The invention is explained below by way of the following examples, which are to be construed as merely illustrative and not limitative for the scope of the invention.

### Examples

### Materials and Methods

The method of the invention (MM or ADHERE) and the prior art procedure (NORMAL) were compared in terms of their ability to purify immunoglobulins from a preparation of the MB12 and MB11 antibodies. As previously mentioned, any reference to MB12 is to be understood as pembrolizumab, whereas references to the MB11 antibody has to be understood as nivolumab.

The workflow of the NORMAL process was composed by:
1. Capture step by protein A affinity (AC);
2. Viral inactivation by low pH (VI);
3. Anion exchange chromatography (AEX);
4. Cation Exchange chromatography (CEX), and
5. Ultrafiltration Diafiltration (UF-DF).

The workflow of the Mixed-Mode process (MM) was the following:
1. Capture step by protein A affinity (AC);
2. Viral inactivation by low pH (VI);
3. Multimodal chromatography (MM);
4. Ultrafiltration-Diafiltration (UF-DF).

The AC was performed with the parameters as defined in Table 1 and buffers as defined in Table 2.

TABLE 1
AC materials and initial parameters

| | | |
|---|---|---|
| Step | Normal Process | Adhere Process |

| | |
|---|---|
| Resin | MabSelect PrismA |
| Bed Height (cm) | 10-22 (Target 20) |
| Process Direction | Down Flow |

TABLE 2
AC buffer compositions

| | | |
|---|---|---|
| Step | Normal Process | Adhere Process |

| | |
|---|---|
| Equilibration | Equilibration buffer |
| Wash 1 | High Salt wash |
| Wash 2 | Equilibration Buffer |
| Elution | Low pH |
| Strip | Low pH |
| Neutralization | Equilibration Buffer |
| CIP | Sodium hydroxide |

The buffers and parameters for VI used were as described in Table 3.

TABLE 3
Buffers and parameters for viral inactivation

| | | |
|---|---|---|
| Step | Normal Process | Adhere Process |

| | |
|---|---|
| Low pH titration buffer | Acidic Buffer |
| Neutralization buffer | Basic Buffer |
| Neutralized conductivity adjustment | WFI |
| Low pH target | < 4.00 |
| Holding step (min) | > 30.00 |

The process and hardware parameters as used for AEX are described in Table 4.

### Platform Membrane SartoBind-Q AEX for Remove Impurities

TABLE 4
Process and hardware parameters for AEX in the NORMAL process

| Step | Buffer (CV) |
|---|---|
| Equilibration | Low conductivity buffer |
| Load | Flowthrough mode |
| Membrane | Sarto Bind-Q |

Tables 5 and 6 display the parameters for the NORMAL process CEX and the ADHERE process MM for the MB12 antibody, respectively.

TABLE 5
Process and hardware parameters for CEX in the NORMAL process

| Step | Buffer (CV) |
|---|---|
| Equilibration | Low conductivity buffer |
| Wash 1 | Low conductivity buffer |
| Elution gradient | Low conductivity buffer |
| | High conductivity buffer |
| Strip1 | High conductivity buffer |
| CIP | NaOH |

Hardware Parameters

| | |
|---|---|
| Resin | Cation Exchanger Resin |
| Bed Height (cm) | 10-22 (Target 20) |

TABLE 6
Process and hardware parameters for MM
in the ADHERE process for the MB12 antibody.

| Step | Buffer (CV) |
|---|---|
| Load pH | 6.4-6.6 (Target 6.5) |
| Load conductivity (mS/cm) | 7.0-8.0 (Target 7.5) |
| pH equilibrium | 6.4-6.6 (Target 6.5) |
| Equilibration | 50.00 mM AcOH at pH = 6.00 & |
| | C: 10mS/cm (5) |
| Wash 1 | 50.00 mM AcOH at pH = 6.00 & |
| | C: 10mS/cm (3,5) |
| Elution gradient | 50.00 mM AcOH at pH = 6.00 & |
| | C: 10mS/cm (10) |
| | 50.00 mM AcOH at pH = 4.00 & |
| | C: 10mS/cm (10) |
| Strip1 | 50.00 mM AcOH at pH = 4.00 & |
| | C: 10mS/cm (10) |
| Strip 2 | NaCI 2 M (4) |
| CIP | NaOH 1 M (4) |

Hardware Parameters

| | |
|---|---|
| Resin | Capto Adhere Impres |
| Bed Height (cm) | 10-22 (Target 20) |
| RT(All operations) (min) | ≥ 3 |
| Max pressure (Bar) | ≤ 2.0 |
| Gradient length (CV) | 20 |
| Elution Collect Start (AU) | Start at 0.125 |
| Elution Collect End (AU) | End at 0.125 |
| Max DBC (g/L) | ≤ 35 |

Table 7 describes the process parameters for the MM in the ADHERE process for the MB11 antibody.

TABLE 7
Process and hardware parameters for MM in the ADHERE process for the MB11 antibody.

| STEP | BUFFER (CV) |
|---|---|
| Load pH | 6.4-6.6 (Target 6.5) |
| Load Conductivity (mS/cm) | 6.5-7.5 (Target 7.0) |
| pH Equilibrium | 6.4-6.6 (Target 6.5) |
| Equilibration | 25mM Bis-TRIS + 25mMAcOH PH = 6.50& C: 7 mS/cm (5) |
| Wash 1 | 25mM Bis-TRIS + 25mMAcOH PH = 6.50& C: 7 mS/cm (3) |
| Elution Gradient 1 | 25mM Bis-TRIS + 25mMAcOH PH = 6.50& C: 7 mS/cm (5) |
| | 25mM Bis-TRIS + 25mMAcOH PH = 5.50& C: 7 mS/cm (5) |
| Elution Gradient 2 | 25mM Bis-TRIS + 25mMAcOH PH = 5.50& C: 7 mS/cm (15) |
| | 25mM Bis-TRIS + 25mMAcOH pH = 4.00& C: 7 mS/cm (15) |
| Strip 1 | 25mM Bis-TRIS + 25mMAcOH PH = 4.00& C: 7 mS/cm (3) |
| Strip/CIP | 1M NaOH + 2M NaCl (3) |
| Hardware Parameters | |
| Resin | Capto Adhere Impres |
| Bed Height (cm) | 10-22 (Target 20) |
| RT (All operations) (min) | ≥ 3 |
| Max pressure (Bar) | ≤ 2.0 |
| Gradient Lenght (CV) | 20 |
| Elution Collect Start (AU) | Start at 0.125 |
| Elution Collect End (AU) | End at 0.125 |
| Max DBC (g/L) | ≤ 30 |

Table 8 describes the process parameters for the UF/DF for both NORMAL and ADHERE processes.

TABLE 8
Process parameters for UF/DF.

| Step | Normal & Adhere Process |
|---|---|
| Cassettes | Pellicon 3 Ultracel PLCTK / Sartocon ECO |
| | Hydrosart* |
| Loading Limit (g/m2) | ≤ 600 |
| Total volume of diafiltered | ≥ 10 |
| solution (DV) | |

* Both cassettes show equivalent unit operation performance.

### Results

The NORMAL downstream process for MB12 and MB11 immunoglobulin purification uses clarified unprocessed bulk as starting material and mainly comprises a set of 3 chromatographic steps (CAF, AEX, CEX) aimed at removing impurities resulting from the process as well as at achieving the optimal product quality within the established ranges. The NORMAL process also contains further steps consisting of a low pH incubation and a nanofiltration step, intended for inactivation and removal of potential virus contaminants. A final Ultrafiltration/diafiltration step is included in order to exchange the buffer so that the product is provided with excipients and the adequate final strength before filling the storage bottles with the bulk drug substance (BDS).

The Multimodal downstream process with Capto Adhere Impres (Cytiva), starts from clarified unprocessed bulk and mainly comprises a set of 2 chromatographic steps (CAF & MM (Multi-Modal)) aimed at removing process and product related impurities as well as at achieving the optimal product quality within the established ranges. The MM process further contains low pH incubation and a nanofiltration steps for inactivation and removal of potential virus contaminants. A final Ultrafiltration/diafiltration step is included in order to attain buffer exchange and to formulate the product with excipients to the final strength before filling the storage bottles with the bulk drug substance (BDS).

During the AC step the appropriate amount of clarified harvest is loaded into the column and followed by a high salt washing step, a second equilibration washing step and an elution step at low pH.

Elution peak is collected based on established criteria based on absorbance. For the VI step, the affinity eluate is diluted to 15 g/L and is titrated to a target pH of <4.00 and incubated >30.00 while gentle mixing. Further the product is neutralized to a target pH of 6.0 (MB12) or 7.0 (MB11). Post neutralization turbidity and flocs formed are removed by depth filtration.

The appropriate amount of VI pool is then passed through the AEX membrane and followed by a washing step. Pool AEX elution is received for in-process control analysis. This step is not carried out in the MM process. For NORMAL process, the AEX pool is loaded into the CEX column and followed by a washing step. Elution is carried using a linear conductivity gradient and fractionated for in-process control analysis and pooled according to the pooling criteria. In the MM process post-VI is loaded into the column and followed by a washing step. Elution is carried using a linear pH gradient with constant conductivity and fractionated for in-process control analysis and pooled according to the pooling criteria.

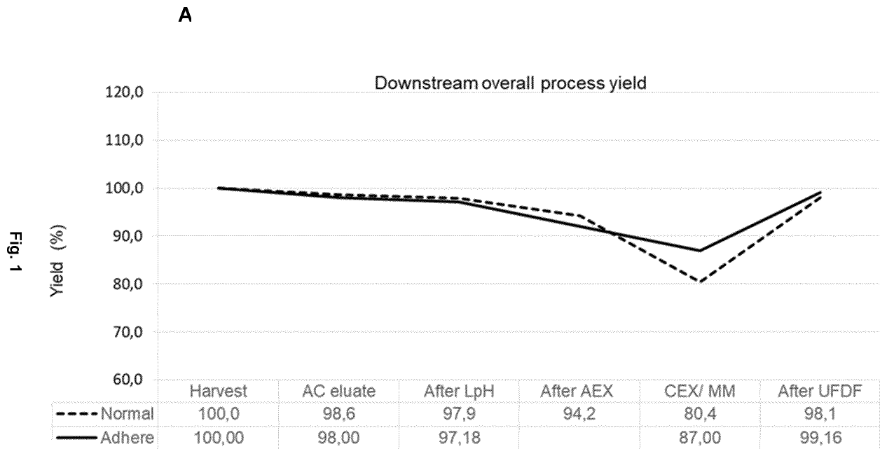

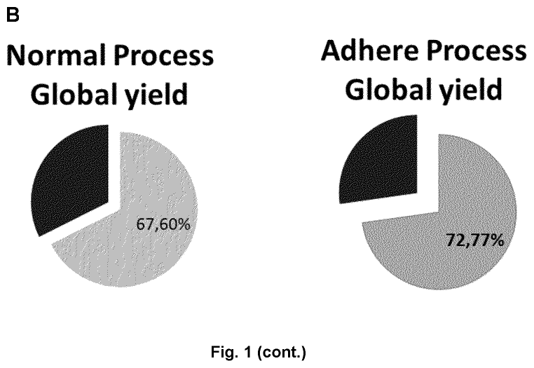

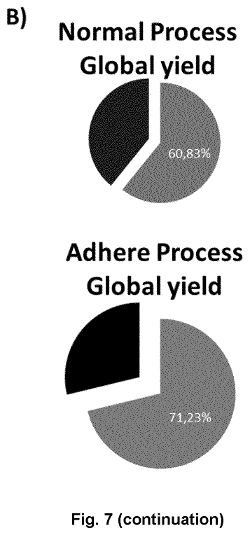

The yield for each process was analyzed after each step of the process as well as globally. As can be seen in FIG. 1A and FIG. 7A, the MM process performed better, obtaining better yield at the step of the MM, with 87% of yield versus the 80.4% obtained after the CEX in the NORMAL process for MB12 and being similar for MB11 (76% for ADHERE vs 75.2% for NORMAL), reaching a global yield of 72.77% in MM versus the 67.6% of the NORMAL process (FIG. 1B) for MB12 and a global yield of 71.23% in MM versus the 60.83% of the NORMAL process (FIG. 7B).

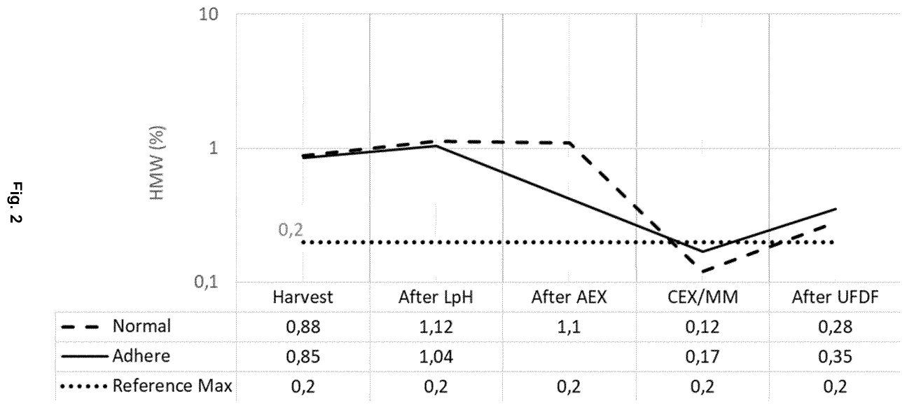

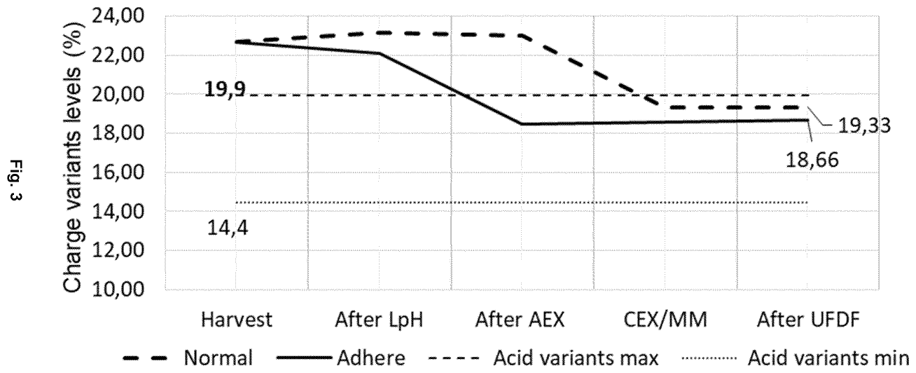

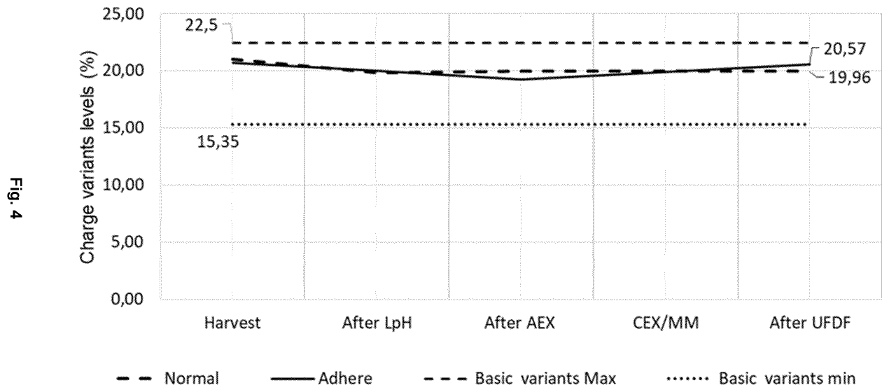

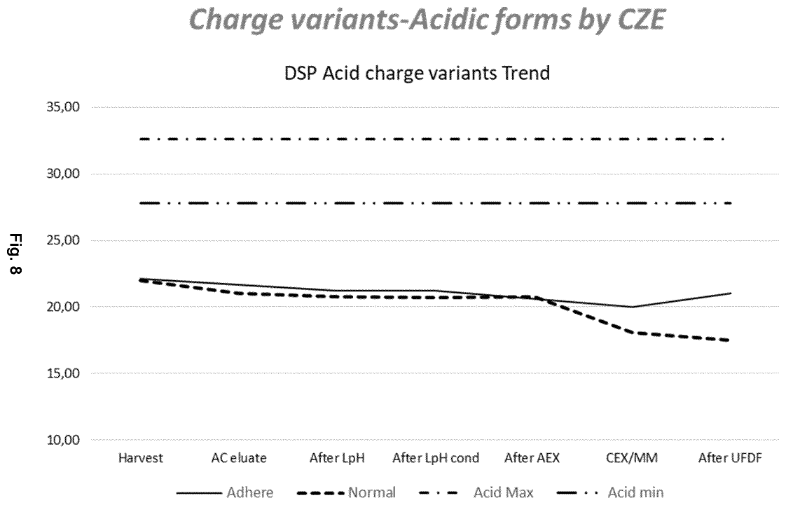

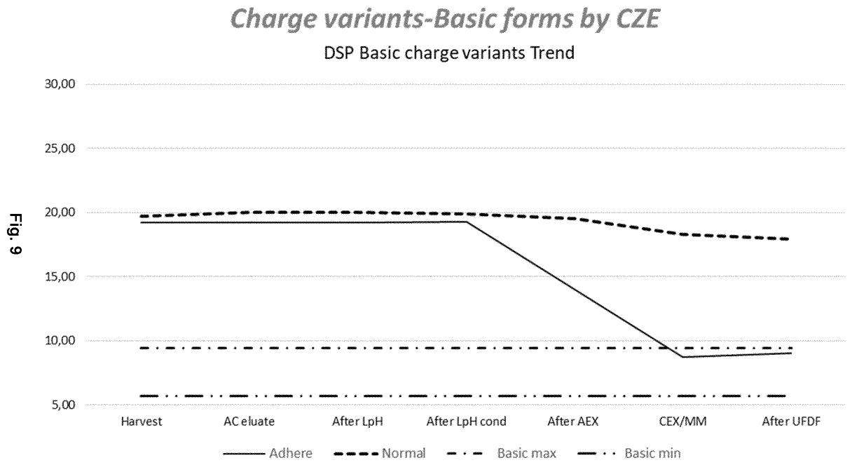

The level of high molecular weight aggregates at each step were then compared for the NORMAL and MM processes. As can be seen in FIG. 2 for MB12, the level of HMW aggregates was comparable between the two processes showing that the MM process is as effective as the NORMAL process at removing HMW aggregates. Likewise, the analysis of the charge variants demonstrated that the MM process performs similarly to the NORMAL process for the both the acidic forms (FIG. 3) for MB12 and FIG. 8 for MB11) and the basic forms (FIG. 4 for MB12). For the case of MB11 the level of charge variants-basic forms following the ADHERE process is employed falls between the established RMP whereas following the NORMAL process it was above the maximum range value of RMP (FIG. 9).

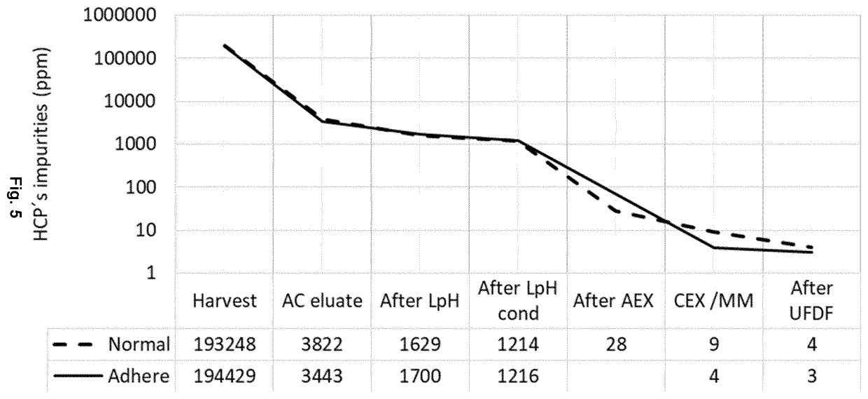

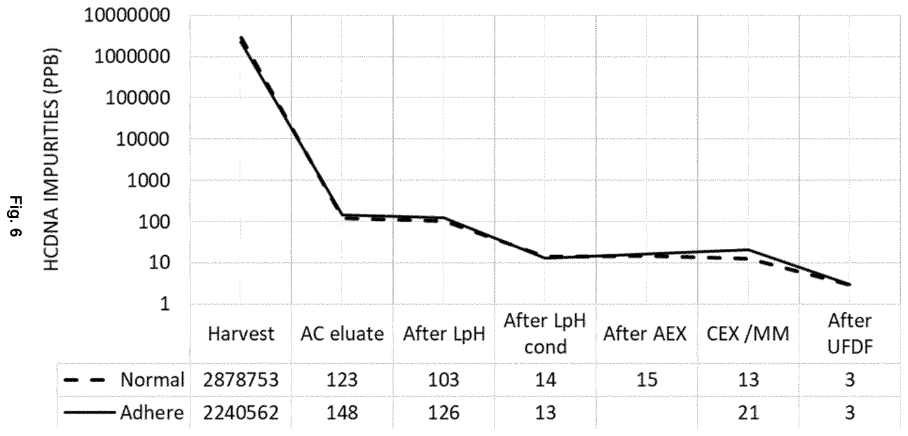

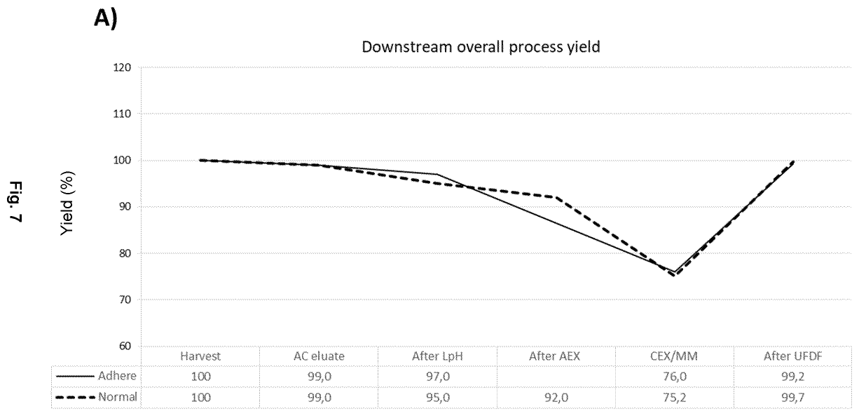

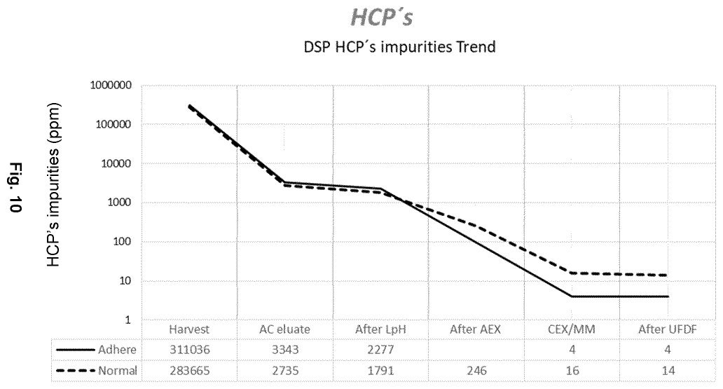

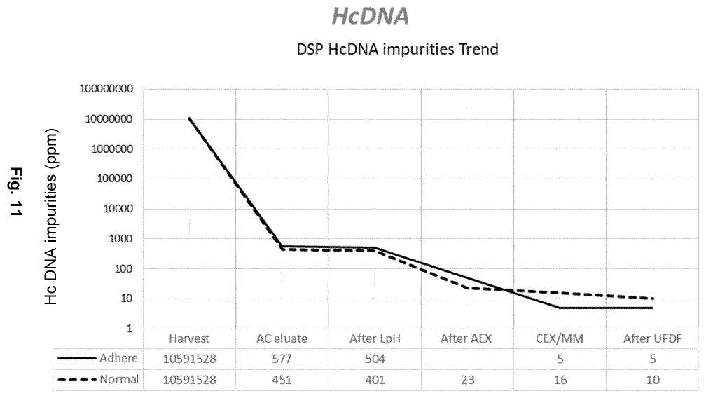

Finally, both processes were analyzed in terms of their capacity of eliminating contaminants from the input solution (host cell proteins or HCPs and host cell DNA or HCDNA). As can be seen in FIGS. 5 and 6 for MB12 and FIGS. 10 and 11 for MB11, both processes are equally capable of eliminating contaminates from the solution, with the ADHERE process performing slightly better than the NORMAL process in the elimination of host cell proteins and host cell DNA.

In conclusion, we have developed a method for the purification of immunoglobulins which not only performs as well as the currently standard process but results in a higher global yield of immunoglobulin protein.

1-40. (canceled)

41. A method for the purification of immunoglobulin molecules present in an immunoglobulin preparation comprising the following steps:
(i) Adjusting the pH of the preparation to a binding pH value which is different from the pI of the immunoglobulin molecules, thereby providing a net charge to the immunoglobulin molecules;
(ii) Contacting the preparation of step (i) with a multimodal ion exchanger at the pH value used in step (i), wherein said multimodal ion exchanger comprises a ligand having an hydrophobic moiety and a moiety which is charged at the binding pH value, wherein the moiety is positively charged if the net charge of the immunoglobulin molecules is positive or wherein the moiety is negatively charged if the net charge of the immunoglobulin molecules is negative, thereby allowing the immunoglobulins to bind to the multimodal anion exchanger and
(iii) Eluting the immunoglobulins bound to the multimodal ion exchanger using a pH gradient at constant conductivity, thereby obtaining a final purified immunoglobulin preparation.

42. The method according to claim 41 wherein the binding pH differs from the isoelectric point of the immunoglobulins in at least 0.5 pH units.

43. The method according to claim 41 wherein the binding pH value is between about 1 to about 12.

44. The method according to claim 41 wherein the conductivity of the preparation in step (i) is between about 5.0 to about 15.0 mS/mc.

45. The method according to claim 44 wherein the conductivity of the preparation in step (i) is about 10.0 mS/mc.

46. The method according to claim 41 wherein the multimodal ion exchanger is a multimodal anion exchanger or a multimodal cationic exchanger.

47. The method according to claim 46 wherein the multimodal anion exchanger is selected from the group consisting of: 4-Mercaptoethylpyridine (4-MEP), tryptophan, phenylpropylamine, hexylamine, N,N-dimethyl-2-phenoxyethan-1-amine, and N-benzyl-N-methyl ethanolamine.

48. The method according to claim 46 wherein the multimodal cationic exchanger is selected from the group consisting of: Sulphonic acid, Sulfopropyl (SP), Methyl sulfonate(S) and Carboxymethyl (CM).

49. The method according to claim 41 wherein the adjusting step (i) is carried so that the binding pH value provides a positive net charge to the immunoglobulin molecules and wherein the contacting step (ii) comprises the steps of
(i) Equilibrating the multimodal ion exchanger with a loading buffer by allowing the loading buffer to flow through the multimodal ion exchanger wherein the pH of the loading buffer is of between about pH 1.0 to about pH 12.0 and has a conductivity of between about 0.1 mS/mc to about 30 mS/mc;
(ii) Loading the multimodal ion exchanger with the immunoglobulin preparation by allowing the immunoglobulin preparation to flow through the multimodal ion exchanger wherein the concentration of immunoglobulin is less than about 100 g/L, and, optionally,
(iii) Washing the multimodal ion exchanger by allowing a washing buffer to flow through the multimodal anion exchanger wherein the washing buffer has a pH of between about pH 1.0 to about pH 12.0 and a conductivity of between about 0.1 mS/mc to about 30 mS/mc.

50. The method according to claim 41 wherein the pH gradient during the elution step (iii) has a starting pH of about 8.0, and an end pH of about 2.0, or a starting pH of about 6.0 and an end pH of about 4.0.

51. The method according to any of claim 41 wherein the conductivity during the elution step (iii) is maintained at a constant value of between about 1.0 mS/cm to about 30.0 mS/cm.

52. The method according to claim 41 wherein the immunoglobulin molecules in the preparation consist essentially of immunoglobulin G or immunoglobulin G4.

53. The method according to claim 52 wherein the immunoglobulin G4 is Pembrolizumab or Nivolumab.

54. The method according to claim 41 further comprising a step of viral inactivation of the immunoglobulin preparation at low pH prior to step (i).

55. The method according to claim 54 wherein the viral inactivation at low pH comprises bringing the immunoglobulin preparation to a pH value of between about 2.0 to about 4.0.

56. The method according to claim 41 further comprising a step prior to step (i) whereby the immunoglobulin preparation is enriched in immunoglobulin molecules by affinity chromatography with an immobilized immunoglobulin specific ligand.

57. The method according to claim 56 wherein the solution obtained after the affinity chromatography step is characterized by having a pH at between about 2.0 to about 12.0, a conductivity of between about 1.0 ms/mc to about 30.0 ms/mc and an immunoglobulin concentration of between about 1.0 g/L to about 100.0 g/L.

58. The method according to claim 41 further comprising a step of ultrafiltration/diafiltration after the elution step (iii), wherein the ultrafiltration/diafiltration is carried out using a filter having a cut-off of 30 kDa and wherein the recovered fraction is the retentate.

59. The method according to claim 58 wherein the eluate obtained after the ultrafiltration/diafiltration is characterized by having a pH of between 2 and 8, a conductivity of between 100 ms/mc and 1500 ms/mc, and an immunoglobulin concentration of between 1.0 g/L and 100.0 g/L.

60. An immunoglobulin obtained by the method according to claim 41.
